(19) United States
(12) Patent Application Publication
(10) Pub. No.: US 2022/0047409 A1
(43) Pub. Date: Feb. 17, 2022

(54) SYSTEMS AND METHODS FOR POSTOPERATIVE SOFT TISSUE CARE

(71) Applicant: MIRRORME3D, New York, NY (US)

(72) Inventors: Jordan Mills, New York, NY (US); Oren Tapper, New York, NY (US); John Layke, New York, NY (US)

(21) Appl. No.: 17/433,871

(22) PCT Filed: Feb. 26, 2020
(86) PCT No.: PCT/US20/19959

§ 371 Date: Aug. 25, 2021

Related U.S. Application Data
(60) Provisional application No. 62/810,451, filed on Feb. 26, 2019.

(51) Int. Cl. A61F 5/08; A61F 5/03; G06F 30/10; G16H 10/60; B33Y 50/00; B33Y 80/00; B29C 64/386

(52) U.S. Cl. CPC A61F 5/08; A61F 5/03; G06F 30/10; G06F 2113/10; B33Y 50/00; B33Y 80/00; B29C 64/386; G16H 10/60

(57) ABSTRACT

Described herein are systems and methods for postoperative soft tissue care that can be effective to control swelling and/or support an intended outcome following a cosmetic or reconstructive procedure.

### RELATED APPLICATIONS

This application claims the benefit of U.S. Provisional Application No. 62/810,451, filed on Feb. 26, 2019, which is incorporated by reference herein in its entirety.

### BACKGROUND

The purpose of postoperative soft tissue care immediately following an operation or procedure is to promote healing while mitigating or diminishing potential complications which can arise during the postoperative period and can prevent the patient from receiving an optimal outcome. Currently, surgeons rely on several methods for immediate postoperative care of soft tissue. These methods include taping, thermoplastic splints, extended thermoplastic splints, and fabric or padded aluminum splints.

### SUMMARY

Various embodiments of the present invention provide systems and methods for postoperative soft tissue care that can be effective to control swelling and/or support an intended outcome (intended postoperative shape/structure/position or progress thereto) via one or more splints configured for use after a cosmetic procedure or a reconstructive procedure is completed.

In some embodiments, the invention provides a computer-implemented method for producing patient-specific splints for postoperative soft tissue care, comprising: receiving, by a computer platform, patient data for a patient including patient-specific 3D imaging data; processing, by the computer platform, the patient data to generate a 3D data file of an intended outcome for a procedure on an anatomy of the patient, wherein the 3D data file is based on the patient-specific 3D imaging data; determining, by the computer platform, a shape for one or more patient-specific splints, wherein the shape of each splint is based on the 3D data file and user input for a plurality of predetermined variables including type of procedure, type of anatomical change, and conditions of the patient and the anatomy prior to the procedure; and forming the one or more patient-specific splints using one or more additive manufacturing technologies or vacuum molding techniques, responsive to the shape determined by the computer platform based on the 3D data file and the user input, wherein each splint is configured to secure the patient's anatomy at a predetermined stage following the procedure.

In some embodiments, said processing comprises detecting orientation of, applying landmarks to, and assigning regions and subregions to the patient-specific 3D imaging data.

In some embodiments, said determining comprises determining the shape for at least two splints, including a splint representing the intended outcome and one or more preceding splints modified therefrom to accommodate one or more levels of expected swelling or movement following the procedure.

In some embodiments, the one or more splints are formed using 3D printing.

In some embodiments, the anatomy is a nose and the procedure is a rhinoplasty.

In some embodiments, the anatomy is a breast and the procedure is at least one of a breast augmentation, a breast reduction, and a breast reconstruction.

In some embodiments, the invention provides a patient-specific splint for postoperative soft tissue care, the splint having a shape determined by a computer platform responsive to (i) a 3D data file of an intended outcome for a procedure on an anatomy of a patient, the 3D data file based on patient data including patient-specific 3D imaging data; and (ii) user input for a plurality of predetermined variables including type of procedure, type of anatomical change, and conditions of the patient and the anatomy prior to the procedure.

In some embodiments, the splint is formed using one or more additive manufacturing technologies or vacuum molding techniques, responsive to the shape determined by the computer platform.

In some embodiments, the splint is formed using 3D printing.

In some embodiments, the splint is formed from a biocompatible resin that is clear or translucent in color.

In some embodiments, the splint further comprises an additional material to permit manual manipulation.

In some embodiments, the additional material comprises a self-curing acrylic resin.

In some embodiments, the splint is perforated.

In some embodiments, the splint is configured to apply force to the anatomy affecting at least one of position, rotation, and projection of the anatomy.

In some embodiments, the anatomy is a nose and the procedure is a rhinoplasty.

In some embodiments, the anatomy is a breast and the procedure is at least one of a breast augmentation, a breast reduction, and a breast reconstruction.

In some embodiments, the invention provides a kit of patient-specific splints for postoperative soft tissue care, each splint having a shape determined by a computer platform responsive to (i) a 3D data file of an intended outcome for a procedure on an anatomy of a patient, the 3D data file based on patient data including patient-specific 3D imaging data; and (ii) user input for a plurality of predetermined variables including type of procedure, type of anatomical change, and conditions of the patient and the anatomy prior to the procedure, the kit comprising at least two patient-specific splints including a splint representing the intended outcome and one or more preceding splints modified therefrom to accommodate one or more levels of expected swelling or movement following the procedure.

In some embodiments, each splint is formed using one or more additive manufacturing technologies or vacuum molding techniques, responsive to the shape determined by the computer platform.

In some embodiments, the anatomy is a nose and the procedure is a rhinoplasty.

In some embodiments, the anatomy is a breast and the procedure is at least one of a breast augmentation, a breast reduction, and a breast reconstruction.

Additional features and advantages of the present invention are described further below. This summary section is meant merely to illustrate certain features of embodiments of the invention, and is not meant to limit the scope of the invention in any way. The failure to discuss a specific feature or embodiment of the invention, or the inclusion of one or more features in this summary section, should not be construed to limit the invention as claimed.

### BRIEF DESCRIPTION OF THE DRAWINGS

The foregoing summary, as well as the following detailed description of certain embodiments, will be better understood when read in conjunction with the appended drawings, in which there are shown certain preferred embodiments. It should be understood, however, that the application is not limited to the precise arrangements and instrumentalities shown. In the drawings:

FIG. 1 is a perspective view showing four illustrative patient-specific rhinoplasty splints in accordance with various embodiments of the invention;

FIG. 2 is a perspective view showing an illustrative patient-specific rhinoplasty splint in accordance with various embodiments of the invention, positioned on a 3D model of the patient's intended outcome;

FIG. 3 is elevational front view of illustrative nose regions that are relevant to the configuration of splints in accordance with various embodiments of the invention;

FIG. 4 is an elevational side view of a splint configured to control postoperative swelling for a patient in accordance with various embodiments of the invention;

FIG. 5 is an elevational side view of a splint configured to control tip shape in accordance with various embodiments of the invention;

FIG. 6 is an elevational side view of a splint configured to control postoperative swelling in the nasal bone regions in accordance with various embodiments of the invention;

FIG. 7 is an elevational side view of a splint provided in a kit of splints configured to control swelling in accordance with various embodiments of the invention;

FIG. 8 is an elevational side view of a splint provided in the same kit as the splint depicted in FIG. 7 in accordance with various embodiments of the invention;

FIG. 9 is an elevational side view of a splint provided in the same kit as the splints depicted in FIG. 7 and FIG. 8 in accordance with various embodiments of the invention;

FIG. 10 is an elevational front view of a splint provided in a kit of splints configured to center the position of a nose in accordance with various embodiments of the invention;

FIG. 11 is a sectional view of the splint depicted in FIG. 10 taken along line J-J;

FIG. 12 is an elevational front view of a splint provided in the same kit as the splint depicted in FIG. 10 in accordance with various embodiments of the invention;

FIG. 13 is a sectional view of the splint depicted in FIG. 12 taken along line K-K;

FIG. 14 is an elevational front view of a splint provided in the same kit as the splints depicted in FIG. 10 and FIG. 12 in accordance with various embodiments of the invention; and

FIG. 15 is a sectional view of the splint depicted in FIG. 12 taken along line L-L.

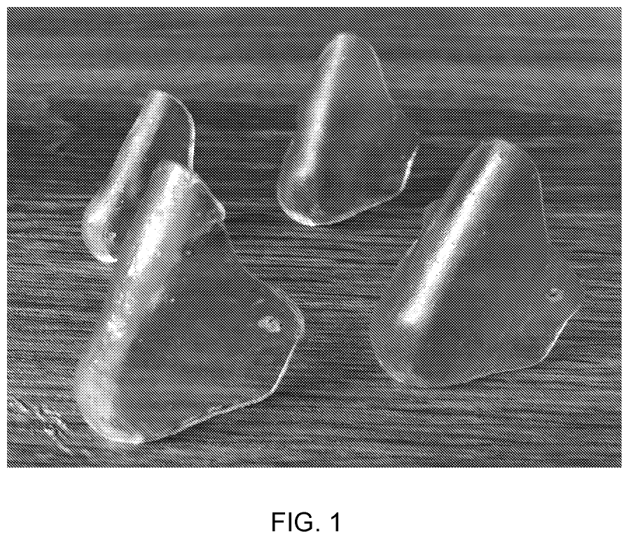

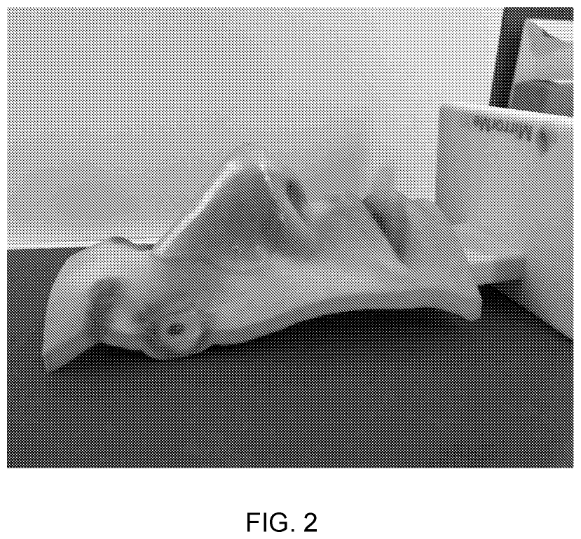

### DETAILED DESCRIPTION

Currently-available systems and methods for postoperative soft tissue care all fall short of actually maintaining shape or structure following surgery for various reasons.

Taping is limited by the skill of the surgeon, the compliance of the patient during the initial days and weeks following surgery, and the mechanical properties of the tape. Tape is not rigid enough to reliably hold a shape during postoperative treatment, which can reduce the overall effect of the surgeon's work. Although immediately utilized by the surgeon following surgery to begin supporting the postoperative healing process, taping is unreliable in its ability to precisely control postoperative swelling (thereby affecting the final shape or structure).

Thermoplastic splints are molded manually based on a best guess for fit and placed on the patient by the surgeon immediately following surgery. They have been shaped (e.g., using scissors or a knife) to be as supportive as possible following surgery with the hope of maintaining the final shape or structure achieved by the surgeon. Although thermoplastic splints provide greater rigidity than tape, they still lack the mechanical properties needed for effectively controlling swelling and shape, since the shape of a thermoplastic splint is limited, for example, by the visual acuity of the surgeon placing the splint and limitations on molding the material. Thermoplastic splints are typically worn for the first week following surgery and may be updated by the surgeon during postoperative appointments.

Extended thermoplastic splints are an improvement over the thermoplastic splints in that they cover more surface area on the patient. However, these are still an unreliable method for controlling swelling, and require additional taping or alterations through the healing process.

Fabric or padded aluminum splints are similar in functionality to the thermoplastic splints. They are useful for keeping the corrected anatomy in a steady position for healing, and are fixed into place as best as possible using hook-and-loop (e.g., VELCRO®) straps, liquid adhesives, or medical tape.

Problems that persist with all of the currently available splinting options include that they do not actively address the effects of postoperative swelling and shape management.

Embodiments of the present invention incorporate a number of important factors identified by the inventors which can affect the shape, size, and mechanical attributes of splints for postoperative soft tissue care. These factors include, but are not limited to: the surgical technique employed by the surgeon (e.g., the type of surgical intervention utilized to achieve the intended outcome, anesthesia, time spent completing the procedure, etc.); the presenting condition of the patient (e.g., the patient's ethnicity, skin thickness, age, the deformity or correction desired, etc.); and the mechanical properties required of the splint (e.g., the applied pressure in designated areas to promote healing, support for shape protection, support for additional movements to reach an optimal outcome, etc.).

Controlling postoperative swelling is important in ensuring that the fully healed anatomy is as close to the surgeon's intended outcome as possible, especially for soft tissue. Most often, splinting and taping are utilized by surgeons during the first week immediately following surgery to protect the recently-corrected shape or structure. Following this first week, surgeons may modify the initial splint to attempt to keep swelling under control and to maintain as close as possible the shape or structure they achieved by the end of surgery. These methods are less than completely satisfactory, however, at least for the reasons highlighted above.

Embodiments of the present invention address the above-identified problems, and provide patient-specific soft tissue postoperative splints that are based on the patient's own medical imaging data (and, thus, the patient's unique anatomical form, also referred to as the presenting or normative condition) and are manufactured to be exact physical matches of the surgeon's intended outcome and/or one or more modifications thereof (e.g., to allow for anticipated edema or other condition(s) occurring immediately after the procedure). As used herein, “intended outcome” refers to the outcome planned by or in collaboration with the surgeon preoperatively (and updated postoperatively if needed), using the patient's medical data (such as, but not limited to, 3D images or scans of the patient's anatomy obtained via 3D imaging methods including stereophotogrammetry, laser scanning, etc.) to determine what the patient will optimally look like after the operation and recovery therefrom. The operation or procedure can be a surgical intervention (invasive or minimally-invasive) or a non-surgical intervention (substantially non-invasive; e.g., a non-surgical rhinoplasty using fillers to achieve symmetry on certain nose deformities).

In various embodiments, after obtaining patient data (such as, but not limited to, patient-specific imaging data, operative plan, and/or data regarding the postoperative outcome), the splint creation process is initiated by ingesting the patient data as detailed further below.

The following inputs may then be provided by the user and may be independent variables in determining the final shape of the splint: (i) type of intervention (surgical or non-surgical), (ii) type of anatomical change, (iii) conditions of patient and patient anatomy (e.g., age, ethnicity, skin thickness, etc.).

Next, the user may input additional dependent variables, which correlate with the independent variables and may or may not further correlate with other dependent variables. Examples of dependent variables include, but are not limited to: (i) hard tissue changes anticipated, (ii) soft tissue changes of internal anatomy, (iii) soft tissue changes of external anatomy, (iv) refinements, enhancements, or other changes to the anatomical shape impacting the aesthetic outcome, (v) compression tolerances as well as site specific compression considerations, (vi) additional considerations which may arise only from intraoperative observation or postoperative observation (e.g., the use of local anesthesia which can result in higher rate of edema post operatively, surgical techniques employed which may result in higher rate of edema post operatively, unknown patient conditions revealed intraoperatively such as unreported scar tissue or trauma, etc.).

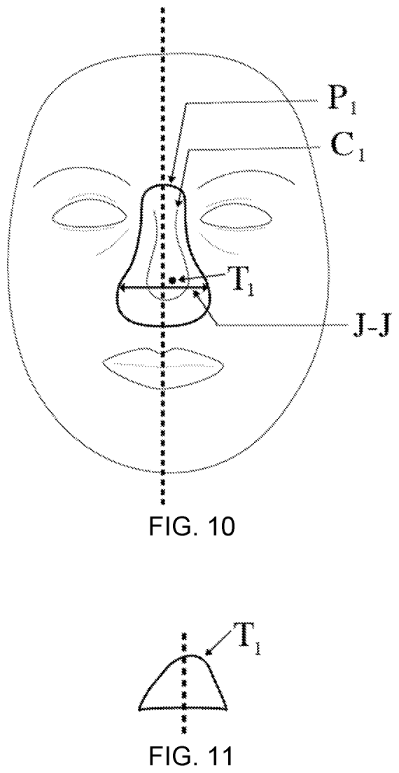

Embodiments of the invention include a methodology for creating one or more patient-specific splints where the shape of the splint is informed by the independent and dependent variables described above and f(Sₓ) is the final shape of the splint. In some embodiments, to begin creating the splint, ingestion/processing of the patient data (also referred to herein as initial input data, including patient-specific 3D imaging data) includes detecting the orientation of the 3D data, applying landmarks (L) to the 3D data (as illustrated with the reference letter T in FIGS. 10-15), and assigning regions (R) to the 3D data which comprise subregions (r). Each subregion (r) is a matrix defined by an absolute value of movement allowable at landmarks within its boundaries.

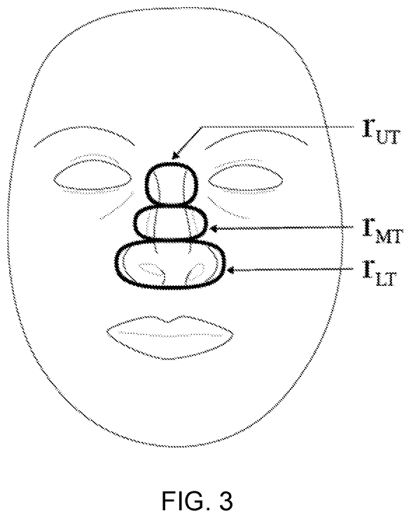

Reference is now made to FIG. 3, which illustrates the subregions (r) as applicable to the region (R) for the creation of the subregions r_{UT}, r_{MT}, and r_{LT} of a rhinoplasty splint and of a rhinoplasty splint kit in accordance with various embodiments of the invention. In FIG. 3, r_{UT} represents the upper third of the region, r_{MT} represents the middle third of the region, and r_{LT} represents the lower third of the region.

The landmarks each denote specific anatomical information including movement (M) allowable which may inform the final shape provided that the landmark is in the area of interest for the splint. Movement allowable is informed by anatomical limitations. For example, landmark L_{RDX} (RDX referring to the radix) is defined as:

L_{RDX}=(Lₓ*Mₓ)(L_{y}*M_{y})*M_{z})

The regions each denote the type of movement allowable in that region and may be related to the landmarks in that the landmarks can be used to determine boundaries of regions and/or minima or maxima information for the region.

Regions provide compression information in addition to total movement allowed in terms of configuration of the one or more splints created. Further, splints' shape boundaries are defined by regions. For example, with reference to FIG. 3, the nose region is defined as:

R=r_{UT},r_{MT},r_{LT}

It should be noted some landmarks may be independent of regions in terms of variable weight in f(Sₓ). Further, any regions outside the area of interest may be removed.

In some embodiments, after initial ingestion of the patient data, modifications to achieve the intended outcome may be input by the user or confirmed by user after detection. These modifications comprise the independent and dependent variables described above where each type of modification may impact the final shape of the splint. Upon receiving all inputs, the final shape of the splint, f(Sₓ), is determined.

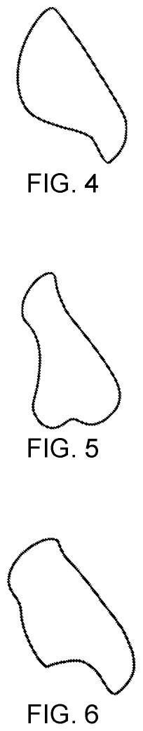

Reference is now made to FIG. 4, which depicts a splint configured to accommodate for a higher rate of swelling due to surgical invention at the dorsum and nasal bones on a patient with higher skin thickness. Upon ingesting/processing the initial input data, the skin thickness results in a greater total volume to accommodate postoperative swelling. The elongated sides in the final shape of the splint are due to both the impact of the skin thickness and the surgical intervention, as the patient may experience a high rate of swelling and the elongated sides can provide a better fit based on these variables.

Reference is now made to FIG. 5, which depicts a splint configured to accommodate for prolonged swelling in the tip of the nose. The final shape of this splint is determined for a primary rhinoplasty with significant tip refinement wherein the patient presents with normal skin thickness, is 32 years old, and of Caucasian descent. The shape in the lower third of the splint is particularly effective to control and protect the shape of tip during recovery as swelling decreases.

Reference is now made to FIG. 6, which depicts a splint configured to accommodate for significant swelling in the upper two thirds of the nose region, where the swelling observed is so significant that additional compression is required in the upper two thirds, but the lower third of the splint requires less compression in the postoperative care process.

If a splint kit is being generated, the shape produced as f(Sₓ) may be modified further. The modifications may or may not require additional input data. If f(Sₓ) requires no additional modifications, the splint series may be reduced in total volume with consideration for the one or more deformities or defects corrected during the procedure. If modifications are required (e.g., if intraoperative observation presented new information affecting the splint shape), the modification is introduced to f(Sₓ) and the shape of the splint is reconfigured accordingly.

For example, rhinoplasty is a complex surgical procedure which requires maximizing the postoperative care to ensure proper healing. During consultation, the trained professional and/or surgeon will take note of one or more deformities or defects of the patient, such as a dorsal hump and drooping tip. The initial data is ingested/processed resulting in the following expression of the landmarks and regions applicable to this example:

$$f ( Sx ) = R$$

$$R = \begin{matrix}{r}_{UT} ( {L}_{1} , {L}_{2} , {L}_{3} … ) \\ {r}_{MT} ( {L}_{7} , {L}_{8} , {L}_{9} … ) \\ {r}_{LT} ( {L}_{15} , {L}_{16} , {L}_{17} … )\end{matrix}$$

Then the patient's ethnicity (e), skin thickness (t), and age (a) is assessed, for this example, adding additional information to inform the splint shape:

$$R = \begin{matrix}{r}_{UT} ( {E}_{e t a} ) \\ {r}_{MT} ( {E}_{e t a} ) \\ {r}_{LT} ( {E}_{e t a} )\end{matrix}$$

The trained professional or surgeon will determine if the rhinoplasty is a primary or revision surgery and will note both the hard tissue changes and soft tissue changes anticipated. For the example of the dorsal hump and drooping tip deformities, this will require hard tissue changes to reduce the dorsal hump and soft tissue changes to correct the drooping tip. For this example, the dorsal hump will need to be reduced by 2.5 mm and drooping tip will require a tip rotation of 5 degrees and tip projection to increase by 2 mm. During the surgery, the surgeon may elect a certain technique for completing the procedure to produce the desired outcome. For this example, to correct the dorsal hump, the surgeon will perform an osteotomy (OT) to reduce the bone along the dorsum to a more aesthetically pleasing line. For the determination of final splint shape, the osteotomy factors to the variable dorsal hump as this type of intervention can cause additional swelling to landmarks and regions at the site of the osteotomy and the fit of the splint will need to compensate for this. Additionally, to correct the drooping tip, the surgeon will have to repair the lower third of the nose to produce a more aesthetically pleasing tip (tip refinement; TR). This may include reducing the cartilage or other soft tissue in the nose and possibly performing an alar base reduction to create the correct tip rotation and projection.

Therefore, r_{UT}, r_{MT}, and r_{LT} are further weighted by the anticipated swelling (E) at landmarks and regions due to the osteotomy and resulting in additional volume and/or shape change of the splint.

$$f ( Sx ) = R = \begin{matrix}{r}_{UT} ( {L}_{1} , {L}_{2} , {L}_{3} … ) ( {E}_{e t a} ) ( {E}_{OT 1} ) \\ {r}_{MT} ( {L}_{7} , {L}_{8} , {L}_{9} … ) ( {E}_{e t a} ) ( {E}_{OT 1} ) \\ {r}_{LT} ( {L}_{15} , {L}_{16} , {L}_{17} … ) ( {E}_{e t a} ) ( {E}_{TR 1} )\end{matrix}$$

As alar base reductions are often an intraoperative decision, this variable may be input later to create one or more additional splints. Because of the nature of the patient-specific splints, embodiments of the present invention include a kit comprising several splints which are successively worn over time. This example would include at least four splints based on the received input data, f(Sx₂) being an earlier splint in the series. The initial splint would protect the nose during the first week postop, and each of the remaining splints would follow to continue to compress the tip for optimized healing and controlling the shape. Compression (C) requires consideration of global movement possible (G) applied at each subregion (r):

$$f ( Sx ) = R$$

$$R = \begin{matrix}{r}_{UT} ( {E}_{e t a} ) ( {G}_{rUT} * {E}_{OT 1} ) \\ {r}_{MT} ( {E}_{e t a} ) ( {G}_{rMT} * {E}_{OT 1} ) \\ {r}_{LT} ( {E}_{e t a} ) ( {G}_{rLT} * {E}_{TR 1} )\end{matrix}$$

$$f ( {Sx}_{2} ) = {R}_{1}$$

$${R}_{1} = \begin{matrix}{r}_{UT} ( {E}_{e t a} ) C ( {G}_{rUT} ) ( {G}_{rUT} * {E}_{OT 1} ) \\ {r}_{MT} ( {E}_{e t a} ) C ( {G}_{{G}_{rMT}} * {E}_{OT 1} ) \\ {r}_{LT} ( {E}_{e t a} ) C ( {G}_{rLT} * {E}_{TR 1} )\end{matrix}$$

In some embodiments, the result of f(Sx) may be a mesh configuration that may be extruded and prepared for production. The final file may be a 3D file of the splint ready for 3D printing or other additive manufacturing techniques, or vacuum molding.

For example, in breast reconstruction, a unilateral breast reconstruction may consist of rebuilding the breast after mastectomy. During the procedure, the surgeon shapes the new breast utilizing a one or more techniques for reconstruction such as introducing an implant, fat grafting, or utilizing a mesh for support. After the procedure, embodiments of the invention provide a patient-specific splint or splint kit for protecting the breast during the postoperative healing phase. Due to the nature of the soft tissue of the breast, the splint or splint kit may be utilized for several years following the procedure to protect the shape of the breast. Without the splint(s), the breast is prone to sagging, changing shape, asymmetry, and/or other deformities which arise during postoperative healing.

By customizing the splints and manufacturing them in a way that can provide a patient-specific fit, the effects of postoperative swelling can be greatly reduced and more precisely controlled, as compared, for example, to moldable thermoplastic splints.

Moreover, by utilizing splints formed based on the requirements of each individual patient to achieve an optimal outcome, as swelling decreases, the desired anatomical shape or structure can be further molded during the weeks immediately following the procedure. This is due to the amount of volume caused by postoperative swelling within the soft tissue. Because soft tissue has been shown to recover relatively slowly from swelling, it is particularly important to protect the desired shape or structure created during the procedure by the surgeon or trained professional. For example, during the weeks immediately post operation or procedure, the cartilaginous anatomy, such as the tip of the nose or the reconstructed breast, is still healing and accepting of additional shaping or molding. Splints according to embodiments of the present invention, whether designed singularly or as part of a kit, can provide continue support for shaping.

Soft tissue postoperative splints according to embodiments of the present invention are custom manufactured preoperatively or postoperatively for each patient based on a simulated outcome produced in collaboration with the surgeon or trained professional, and can control swelling more effectively than currently available splinting options, which are manually formed postoperatively based on an estimation by eye of whatever shape is present at the end of the operation. Splints according to embodiments of the present invention may be created, for example, using 3D printing or other additive manufacturing technology/techniques, vacuum molding techniques, and/or other technologies currently known or to be developed in the future.

In some embodiments, the invention provides patient-specific rhinoplasty splints, which may be configured to protect the tip of the nose postoperatively, where much of the swelling (edema) is located. When not controlled, as swelling decreases, the shape and/or location of the tip can change during the healing process, for a year or more post operation.

In other embodiments, the invention provides patient-specific splints for use on different types of soft tissue anatomy, such as, but not limited to, the breast(s).

In some embodiments, the invention provides splints formed from a plastic material such as a biocompatible resin. In some embodiments, the plastic is clear or translucent in color. Clear plastic can provide a more discrete look and can be perforated or ventilated in various ways to allow moisture to escape. Perforation is optional, and may be selected, for example, based on skin type and/or physician preference. In other embodiments, splints according to embodiments of the present invention may be formed from another suitable material.

In some embodiments, the invention provides splints manufactured using other malleable/moldable material(s) in addition to the biocompatible plastic, the additional material(s) allowing for table-side manipulation when necessary (e.g., when unexpected intraoperative decisions must be made in consideration of the surgical intervention). For example, splints according to embodiments of the present invention may include a material such as a self-curing acrylic resin that can be slightly altered (e.g., by wetting) for last-minute changes if the planned outcome has not been fully achieved or if additional changes need to be made on the operating table in response to unobservable conditions within the patient's anatomy. For example, a plastic surgeon performing a rhinoplasty may not always have a CT scan performed/available, and therefore they may lack information about the internal conditions of the patient's nose until they are performing the surgery. They may find unreported scar tissue or other conditions which can affect the final outcome or prevent them from achieving the initially planned outcome, thus adjusting the intraoperative plan which then requires immediate modifications for the splint to be fully effective. Thus, splints according to embodiments of the present invention may include one or more materials that allow for surgeons to adjust the splints table side (or chair side) as needed.

In some embodiments, more than one splint according to the present invention may be provided as a series, for example, in a kit that includes at least two different sizes (e.g., 3-5 sizes, although other numbers may be used) of splints configured to support, protect, move, compress, and/or position the patient's anatomy progressively during the healing process. Each fit may be determined by types of alterations to be done and the splints may be strategically reduced in size, shape, volume, or combination thereof, in part or in whole, over the course of postoperative wear. For example, reducing the size of at least part of the splint over the course of wear can progressively compress the patient's anatomy, and the swelling, and thus can control postoperative shape more accurately. Additionally, the splints can be modified proportionally to the amount of expected swelling to further control the final shaping of the anatomy where soft tissue is prominent.

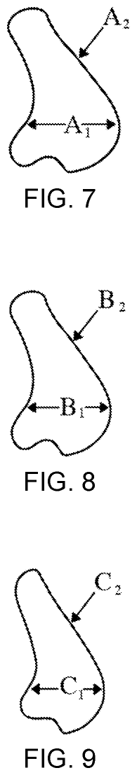

Reference is now made to FIGS. 7-9, which depict an illustrative splint kit comprising three postoperative soft tissue splints for the example of a rhinoplasty correcting the dorsal hump and drooping tip. Each splint in the kit is configured to accommodate swelling in two regions of the area of interest (regions depicted in FIG. 3): the region which encompasses the dorsum (location of the dorsal hump deformity; marked “A₂,” “B₂,” and “C₂” in FIG. 7, FIG. 8, and FIG. 9, respectively) and the region which encompasses the tip (location of the drooping tip deformity; marked “A₁,” “B₁,” and “C₁” in FIG. 7, FIG. 8, and FIG. 9, respectively).

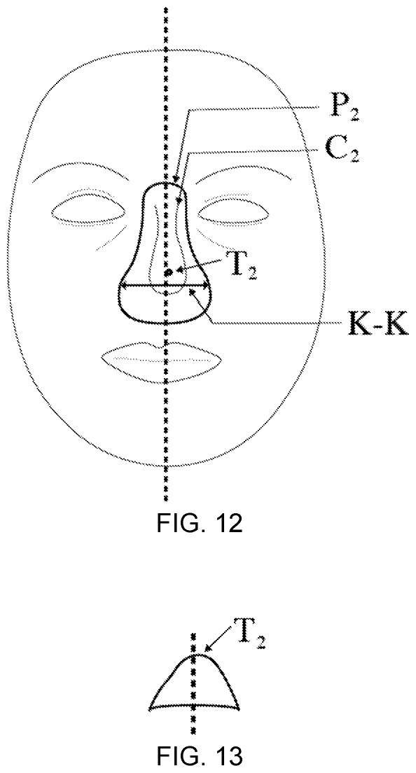

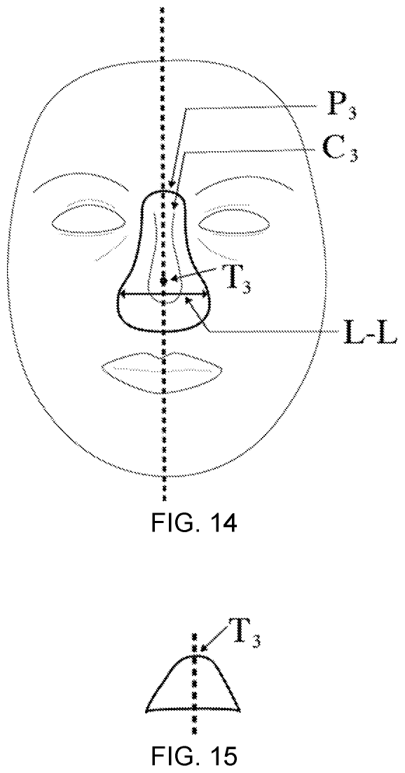

Reference is now made to FIGS. 10-15, which depict an illustrative splint kit where each splint is configured to apply force to move the tip (marked at T₁, T₂, T₃) towards the midline (the vertical dashed line) over several weeks of postoperative wear. FIG. 10 shows the first splint of the kit being utilized to move the tip (T₁) towards the midline (vertical dashed line). The perimeter of the splint (P₁) covers the region affected with the contour line (C₁) and shows the splint shape. FIG. 11 is a sectional view at J-J of the first splint further depicting the tip (T₁) location at the onset of the patient using the first splint of the splint kit. FIG. 12 shows the second splint of the kit being utilized to move the tip (T₂) towards the midline (vertical dashed line). The perimeter of the splint (P₂) covers the region affected with the contour line (C₂) and shows the splint shape. FIG. 13 is a sectional view at K-K of the second splint further depicting the tip (T₂) location at the onset of the patient using the second splint of the splint kit. FIG. 14 shows the third splint of the kit being utilized to move the tip (T₃) towards the midline (vertical dashed line). The perimeter of the splint (P₃) covers the region affected with the contour line (C₃) and shows the splint shape. FIG. 15 is a sectional view at L-L of the third splint further depicting the tip (T₃) location at the onset of the patient using the third splint of the splint kit.

In an actual patient use of the kit of splints depicted in FIGS. 10-15, the amount of movement achieved at T₁ was found to be 2.0 mm, the amount of movement achieved at T₂ was found to be 1.34 mm, and the amount of movement achieved at T₃ was found to be 0.84 mm. However, it will be understood that each of these amounts will vary on a patient by patient basis due to the variables inherent in patient-specific surgical procedures and care.

While there have been shown and described fundamental novel features of the invention as applied to the preferred and illustrative embodiments thereof, it will be understood that omissions and substitutions and changes in the form and details of the disclosed invention may be made by those skilled in the art without departing from the spirit of the invention. Moreover, numerous modifications and changes may readily occur to those skilled in the art. For example, various features and structures of the different embodiments discussed herein may be combined and interchanged. Hence, it is not desired to limit the invention to the exact construction and operation shown and described and, accordingly, all suitable modification equivalents may be resorted to falling within the scope of the invention as claimed. It is the intention, therefore, to be limited only as indicated by the scope of the claims appended hereto.

What is claimed is:

1. A computer-implemented method for producing patient-specific splints for postoperative soft tissue care, comprising:
receiving, by a computer platform, patient data for a patient including patient-specific 3D imaging data;
processing, by the computer platform, the patient data to generate a 3D data file of an intended outcome for a procedure on an anatomy of the patient, wherein the 3D data file is based on the patient-specific 3D imaging data;
determining, by the computer platform, a shape for one or more patient-specific splints, wherein the shape of each splint is based on the 3D data file and user input for a plurality of predetermined variables including type of procedure, type of anatomical change, and conditions of the patient and the anatomy prior to the procedure; and
forming the one or more patient-specific splints using one or more additive manufacturing technologies or vacuum molding techniques, responsive to the shape determined by the computer platform based on the 3D data file and the user input, wherein each splint is configured to secure the patient's anatomy at a predetermined stage following the procedure.

2. The method of claim 1, wherein said processing comprises detecting orientation of, applying landmarks to, and assigning regions and subregions to the patient-specific 3D imaging data.

3. The method of claim 1, wherein said determining comprises determining the shape for at least two splints, including a splint representing the intended outcome and one or more preceding splints modified therefrom to accommodate one or more levels of expected swelling or movement following the procedure.

4. The method of claim 1, wherein the one or more splints are formed using 3D printing.

5. The method of claim 1, wherein the anatomy is a nose and the procedure is a rhinoplasty.

6. The method of claim 1, wherein the anatomy is a breast and the procedure is at least one of a breast augmentation, a breast reduction, and a breast reconstruction.

7. A patient-specific splint for postoperative soft tissue care, the splint having a shape determined by a computer platform responsive to (i) a 3D data file of an intended outcome for a procedure on an anatomy of a patient, the 3D data file based on patient data including patient-specific 3D imaging data; and (ii) user input for a plurality of predetermined variables including type of procedure, type of anatomical change, and conditions of the patient and the anatomy prior to the procedure.

8. The patient-specific splint of claim 7, wherein the splint is formed using one or more additive manufacturing technologies or vacuum molding techniques, responsive to the shape determined by the computer platform.

9. The patient-specific splint of claim 8, wherein the splint is formed using 3D printing.

10. The patient-specific splint of claim 7, wherein the splint is formed from a biocompatible resin that is clear or translucent in color.

11. The patient-specific splint of claim 10, wherein the splint further comprises an additional material to permit manual manipulation.

12. The patient-specific splint of claim 11, wherein the additional material comprises a self-curing acrylic resin.

13. The patient-specific splint of claim 7, wherein the splint is perforated.

14. The patient-specific splint of claim 7, wherein the splint is configured to apply force to the anatomy affecting at least one of position, rotation, and projection of the anatomy.

15. The patient-specific splint of claim 7, wherein the anatomy is a nose and the procedure is a rhinoplasty.

16. The patient-specific splint of claim 7, wherein the anatomy is a breast and the procedure is at least one of a breast augmentation, a breast reduction, and a breast reconstruction.

17. A kit of patient-specific splints for postoperative soft tissue care, each splint having a shape determined by a computer platform responsive to (i) a 3D data file of an intended outcome for a procedure on an anatomy of a patient, the 3D data file based on patient data including patient-specific 3D imaging data; and (ii) user input for a plurality of predetermined variables including type of procedure, type of anatomical change, and conditions of the patient and the anatomy prior to the procedure, the kit comprising at least two patient-specific splints including a splint representing the intended outcome and one or more preceding splints modified therefrom to accommodate one or more levels of expected swelling or movement following the procedure.

18. The kit of claim 17, wherein each splint is formed using one or more additive manufacturing technologies or vacuum molding techniques, responsive to the shape determined by the computer platform.

19. The kit of claim 17, wherein the anatomy is a nose and the procedure is a rhinoplasty.

20. The kit of claim 17, wherein the anatomy is a breast and the procedure is at least one of a breast augmentation, a breast reduction, and a breast reconstruction.
